(19) United States
(12) Patent Application Publication
(10) Pub. No.: US 2008/0124376 A1
(43) Pub. Date: May 29, 2008

(54) OCULAR DEVICES AND METHODS OF MAKING AND USING THEREOF

(75) Inventors: John Dallas PRUITT, Suwanee, GA (US); Lynn Cook WINTERTON, Alpharetta, GA (US); John Martin LALLY, Laguna Niguel, CA (US)

Correspondence Address: GARDNER GROFF GREENWALD & VILLANUEVA. PC, 2018 POWERS FERRY ROAD, SUITE 800, ATLANTA, GA, 30339, US

(73) Assignee: NOVARTIS AG, Basel (CH)

(21) Appl. No.: 11/934,817

(22) Filed: Nov. 5, 2007

Related U.S. Application Data
(60) Provisional application No. 60/864,428, filed on Nov. 6, 2006.

(51) Int. Cl. A61K 9/10; A61K 31/35; A61K 31/445; A61K 31/453; A61K 31/728; A61P 27/00

(52) U.S. Cl. 424/427; 514/456; 514/324; 514/291; 514/53

(57) ABSTRACT

Described herein are stable ocular devices that immobilize and deliver bioactive agents to the eye over sustained periods of time. Also described herein are methods of making and using the ocular devices.

### CROSS-REFERENCE TO RELATED APPLICATION

This application claims the priority benefit of U.S. Provisional Patent Application Ser. No. 60/864,428, filed Nov. 6, 2006, which is hereby incorporated by reference herein.

### BACKGROUND

Controlled- or sustained-released drug-delivery systems are well known in the pharmaceutical industry. However, this type of technology is not well known in the contact lens industry. Industries have tried to overcome this problem by “loading” the polymerized article after-the-fact. This is accomplished by swelling the article in an appropriate solvent (much like in an extraction step) and then solubilizing the active compound/ingredient into that same solvent. After equilibrium, the loaded-product is removed from the solvent, allowed to dry to remove the solvent, or the solvent is exchanged with a solvent that does not solvate the loaded-active or swell the polymer matrix. This results in a dry-loaded article that is capable of releasing the desired compound or ingredient.

There are several disadvantages associated with this “loading” process. First, it requires many additional steps, which can increase production costs. Second, loading efficiency largely depends on the solubilization parameter of the compound or ingredient to be loaded on the lens. Third, the article must be dried or exposed to solvent exchange. This is difficult to accomplish in view of current lens packaging systems, where hydrogel contact lenses are stored in a packaging solution (i.e., a hydrated state). Finally, once the article is hydrated, the release mechanism is activated and the loaded material is released. Since hydrogel contact lenses are stored in a packaging solution, most if not all of the loaded compound is already released in the packaging solution.

Therefore, there exists a need for ocular devices such as, for example, contact lenses, capable of delivering an active compound in a sustainable manner over an extended period of time. The devices described herein release one or more bioactive agents when the device comes into contact with one or more tear components produced by the eye. Thus, the tear components “trigger” the release of the bioactive agent, which helps control the rate of release of the bioactive agent from the device, particularly over extended periods of time.

### SUMMARY

Described herein are stable ocular devices that immobilize and deliver bioactive agents to the eye over sustained periods of time. Also described herein are methods of making and using the ocular devices. The advantages of the invention will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below.

FIG. 1 shows the release pattern of 50 kDa, 100 kDa, and 1 M Da hyaluronan from a Nelfilcon matrix.

FIG. 2 shows the release pattern of 1 M Da hyaluronan at various concentrations from a Nelfilcon matrix.

FIG. 3 shows the heat stability of lens composed of Nelfilcon with hyaluronan.

FIG. 4 shows the release pattern of Rose Bengal from Nelfilcon lenses placed in saline solutions (PBS) and lysozyme.

### DETAILED DESCRIPTION

Before the present compounds, compositions, and methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

It must be noted that, as used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

“Optional” or “optionally” means that the subsequently described event or circumstance can or cannot occur, and that the description includes instances where the event or circumstance occurs and instances where it does not. For example, the phrase “optionally substituted lower alkyl” means that the lower alkyl group can or cannot be substituted and that the description includes both unsubstituted lower alkyl and lower alkyl where there is substitution.

Unless defined otherwise, all technical and scientific terms used herein have the same meaning as commonly understood by one of ordinary skill in the art to which this invention belongs. Generally, the nomenclature used herein and the laboratory procedures are well known and commonly employed in the art. Conventional methods are used for these procedures, such as those provided in the art and various general references. The nomenclature used herein and the laboratory procedures described below are those well known and commonly employed in the art. As employed throughout the disclosure, the following terms, unless otherwise indicated, shall be understood to have the following meanings.

A “hydrogel” refers to a polymeric material that can absorb at least 10 percent by weight of water when it is fully hydrated. A hydrogel material can be obtained by polymerization or copolymerization of at least one hydrophilic monomer in the presence of or in the absence of additional monomers and/or macromers or by crosslinking of a prepolymer.

A “silicone hydrogel” refers to a hydrogel obtained by copolymerization of a polymerizable composition comprising at least one silicone-containing vinylic monomer or at least one silicone-containing macromer or a silicone-containing prepolymer.

“Hydrophilic,” as used herein, describes a material or portion thereof that will more readily associate with water than with lipids.

The term “fluid” as used herein indicates that a material is capable of flowing like a liquid.

A “monomer” means a low molecular weight compound that can be polymerized actinically or thermally or chemically. Low molecular weight typically means average molecular weights less than 700 Daltons.

As used herein, “actinically” in reference to curing or polymerizing of a polymerizable composition or material or a matrix-forming material means that the curing (e.g., crosslinked and/or polymerized) is performed by actinic irradiation, such as, for example, UV irradiation, ionized radiation (e.g. gamma ray or X-ray irradiation), microwave irradiation, and the like. Thermal curing or actinic curing methods are well-known to a person skilled in the art.

A “vinylic monomer,” as used herein, refers to a low molecular weight compound that has an ethylenically unsaturated group and can be polymerized actinically or thermally. Low molecular weight typically means average molecular weights less than 700 Daltons.

The term “ethylenically unsaturated group” or “olefinically unsaturated group” is employed herein in a broad sense and is intended to encompass any groups containing at least one C═C group. Exemplary ethylenically unsaturated groups include without limitation acryloyl, methacryloyl, allyl, vinyl, styrenyl, or other C═C containing groups.

A “hydrophilic vinylic monomer,” as used herein, refers to a vinylic monomer that is capable of forming a homopolymer that can absorb at least 10 percent by weight water when fully hydrated. Suitable hydrophilic monomers are, without this being an exhaustive list, hydroxyl-substituted lower alkyl (C₁ to C₈) acrylates and methacrylates, acrylamide, methacrylamide, (lower allyl)acrylamides and -methacrylamides, ethoxylated acrylates and methacrylates, hydroxyl-substituted (lower alkyl)acrylamides and -methacrylamides, hydroxyl-substituted lower alkyl vinyl ethers, sodium vinylsulfonate, sodium styrenesulfonate, 2-acrylamido-2-methylpropanesulfonic acid, N-vinylpyrrole, N-vinyl-2-pyrrolidone, 2-vinyloxazoline, 2-vinyl-4,4′-dialkyloxazolin-5-one, 2- and 4-vinylpyridine, vinylically unsaturated carboxylic acids having a total of 3 to 5 carbon atoms, amino(lower alkyl)—(where the term “amino” also includes quaternary ammonium), mono(lower alkylamino)(lower alkyl) and di(lower alkylamino)(lower alkyl)acrylates and methacrylates, allyl alcohol and the like.

A “hydrophobic vinylic monomer,” as used herein, refers to a vinylic monomer that is capable of forming a homopolymer that can absorb less than 10 percent by weight water.

A “macromer” refers to a medium to high molecular weight compound or polymer that contains functional groups capable of undergoing further polymerizing/crosslinking reactions. Medium and high molecular weight typically means average molecular weights greater than 700 Daltons. In one aspect, the macromer contains ethylenically unsaturated groups and can be polymerized actinically or thermally.

A “prepolymer” refers to a starting polymer that can be cured (e.g., crosslinked and/or polymerized) actinically or thermally or chemically to obtain a crosslinked and/or polymerized polymer having a molecular weight much higher than the starting polymer. A “actinically-crosslinkable prepolymer” refers to a starting polymer which can be crosslinked upon actinic radiation or heating to obtain a crosslinked polymer having a molecular weight much higher than the starting polymer. In accordance with the invention, an actinically-crosslinkable prepolymer is soluble in a solvent and can be used in producing a finished ocular device of optical quality by cast-molding in a mold without the necessity for subsequent extraction.

### I. Ocular Devices and Methods of Making Thereof

Described herein are ocular devices comprising a polymeric matrix and a bioactive agent incorporated within the polymeric matrix, wherein the bioactive agent is released from the polymeric matrix by one or more tear components. As will be discussed in more detail below, the bioactive agent is incorporated throughout the polymeric matrix and immobilized. The bioactive agent is “incorporated within” the polymeric matrix by modifying the properties of the bioactive agent and polymeric matrix such that the bioactive agent and polymeric matrix interact with one another. The interaction between the bioactive agent and polymeric matrix can assume many forms. Examples of such interactions include, but are not limited to, covalent and/or non-covalent interactions (e.g., electrostatic, a hydrophobic/hydrophobic, dipole-dipole, Van der Waals, hydrogen bonding, and the like). Each of these interactions with respect to the selection of the bioactive agent and the polymeric matrix will be discussed below.

The ocular devices produced herein are stable with respect to retaining (i.e., immobilizing) the bioactive agent. The devices described herein are specifically designed to release the bioactive agent when they come into contact with one or more tear components produced by the eye. The tear components “trigger” the release of the bioactive agent and provide for a sustained release of the bioactive agent to the eye. Thus, the ocular device is capable of being induced by one or more tear-component to release of bioactive agent over an extended period of wearing time. In a preferred embodiment, the ocular devices described herein can be stored for extended periods of time in a packaging solution without the bioactive agent leaching from the device to a significant extent (i.e., leaching less than about 20%, less than about 15%, less than about 10%, less than about 8%, preferably less than about 5%, more preferably less than about 2%, even more preferably less than about 1% of the total amount of bioactive agent distributed in the polymer matrix after storing for one year in the packaging solution) into the packaging solution (e.g., saline solution) in the package.

Tear component-induced release of a bioactive agent can be characterized by the following example. Contact lenses with a bioactive agent distributed therein can be soaked in a given volume of a buffered saline (e.g., phosphate buffered saline) and in a given volume of a buffered saline including one or more tear components (e.g., including without limitation, lysozyme, lipids, lactoferrin, albumin, etc.) for a period of time (e.g., 30 minutes, 60 minutes, or 120 minutes). The concentrations of the bioactive agent leached from the lenses into the buffered saline and into the buffered saline having one or more tear components are determined and compared with each other. Where the concentration of the leached bioactive agent in the buffered saline having one or more tear components is at least 10% higher than that in the buffered saline, there is tear component-induced release of the bioactive agent from the lens with the bioactive agent distributed therein.

Described below are the different components used to prepare the ocular devices described herein as well as methods for making the devices. Also described herein are methods for using the devices described herein for delivering one or more bioactive agents to the eye of a subject.

a. Polymeric Matrix

The polymeric matrix used in the devices described herein are prepared from a matrix forming material. The term “matrix-forming material” is defined herein as any material that is capable of being polymerized using techniques known in the art. The matrix-forming material can be a monomer, a prepolymer, a macromolecule or any combination thereof. It is contemplated that the matrix forming material can be modified prior to polymerization or the polymeric matrix can be modified after polymerization of the matrix forming material. The different types of modifications will be discussed below.

In one aspect, the matrix-forming material (prepolymer composition) comprises a prepolymer. For example, a fluid prepolymer composition comprising at least one actinically-crosslinkable prepolymer can be used. The matrix-forming material can be a solution, a solvent-free liquid, or a melt. In one aspect, the fluid prepolymer composition is an aqueous solution comprising at least one actinically-crosslinkable prepolymer. It is understood that the prepolymer composition can also include one or more vinylic monomers, one or more vinylic macromers, and/or one or more crosslinking agents. However, the amount of those components should be low such that the final ocular device does not contain unacceptable levels of unpolymerized monomers, macromers and/or crosslinking agents. The presence of unacceptable levels of unpolymerized monomers, macromers and/or crosslinking agents will require extraction to remove them, which requires additional steps that are costly and inefficient.

The prepolymer composition can further comprise various components known to a person skilled in the art, including without limitation, polymerization initiators (e.g., photoinitiator or thermal initiator), photosensitizers, UV-absorbers, tinting agents, antimicrobial agents, inhibitors, fillers, and the like, so long as the device does not need to be subjected to subsequent extraction steps. Examples of suitable photoinitiators include, but are not limited to, benzoin methyl ether, 1-hydroxycyclohexylphenyl ketone, or Darocure® or Irgacure® types, for example Darocure® 1173 or Irgacure® 2959. The amount of photoinitiator can be selected within wide limits, an amount of up to 0.05 g/g of prepolymer and preferably up to 0.003 g/g of prepolymer can be used. A person skilled in the art will know well how to select the appropriate photoinitiator.

The use of other solvents in combination with water can be used to prepare the matrix-forming material. For example, the aqueous prepolymer solution can also include, for example an alcohol, such as methanol, ethanol or n- or iso-propanol, or a carboxylic acid amide, such as N,N-dimethylformamide, or dimethyl sulfoxide. In one aspect, the aqueous solution of prepolymer contains no further solvent. In another aspect, the aqueous solution of the prepolymer does not contain unreacted matrix-forming material that needs to be removed after the device is formed.

In one aspect, a solution of at least one actinically-crosslinkable prepolymer can be prepared by dissolving the actinically-crosslinkable prepolymer and other components in any suitable solvent known to a person skilled in the art. Examples of suitable solvents are water, alcohols (e.g., lower alkanols having up to 6 carbon atoms, such as ethanol, methanol, propanol, isopropanol), carboxylic acid amides (e.g., dimethylformamide), dipolar aprotic solvents (e.g., dimethyl sulfoxide or methyl ethyl ketone), ketones (acetone or cyclohexanone), hydrocarbons (e.g., toluene), ethers (e.g., THF, dimethoxyethane or dioxane), and halogenated hydrocarbons (e.g., trichloroethane), and any combination thereof.

In one aspect, the matrix-forming material comprises a water-soluble actinically-crosslinkable prepolymer. In another aspect, the matrix-forming material comprises an actinically-crosslinkable prepolymer that is soluble in a water-organic solvent mixture, or an organic solvent, meltable at a temperature below about 85° C., and are ophthalmically compatible. In various aspects, it is desirable that the actinically-crosslinkable prepolymer is in a substantially pure form (e.g., purified by ultrafiltration to remove most reactants for forming the prepolymer). Thus, after polymerization, the device will not require subsequent purification such as, for example, costly and complicated extraction of unpolymerized matrix-forming material. Furthermore, crosslinking of the matrix-forming material can take place absent a solvent or in aqueous solution so that a subsequent solvent exchange or the hydration step is not necessary.

Examples of actinically crosslinkable prepolymers include, but are not limited to, a water-soluble crosslinkable poly(vinyl alcohol) prepolymer described in U.S. Pat. Nos. 5,583,163 and 6,303,687 (incorporated by reference in their entireties); a water-soluble vinyl group-terminated polyurethane prepolymer described in U.S. Patent Application Publication No. 2004/0082680 (herein incorporated by reference in its entirety); derivatives of a polyvinyl alcohol, polyethyleneimine or polyvinylamine, which are disclosed in U.S. Pat. No. 5,849,841 (incorporated by reference in its entirety); a water-soluble crosslinkable polyurea prepolymer described in U.S. Pat. No. 6,479,587 and in U.S. Published Application No. 2005/0113549 (herein incorporated by reference in their entireties); crosslinkable polyacrylamide; crosslinkable statistical copolymers of vinyl lactam, MMA and a comonomer, which are disclosed in EP 655,470 and U.S. Pat. No. 5,712,356; crosslinkable copolymers of vinyl lactam, vinyl acetate and vinyl alcohol, which are disclosed in EP 712,867 and U.S. Pat. No. 5,665,840; polyether-polyester copolymers with crosslinkable side chains which are disclosed in EP 932,635 and U.S. Pat. No. 6,492,478; branched polyalkylene glycol-urethane prepolymers disclosed in EP 958,315 and U.S. Pat. No. 6,165,408; polyalkylene glycol-tetra(meth)acrylate prepolymers disclosed in EP 961,941 and U.S. Pat. No. 6,221,303; crosslinkable polyallylamine gluconolactone prepolymers disclosed in International Application No. WO 2000/31150 and U.S. Pat. No. 6,472,489; and silicone-containing prepolymers are those described in commonly-owned U.S. Pat. Nos. 6,039,913, 7,091,283, 7,268,189 and 7,238,750, and U.S. patent application Ser. No. 09/525,158 filed Mar. 14, 2000 (entitled “Organic Compound”), 11/825,961, 60/869,812 filed Dec. 13, 2006 (entitled “PRODUCTION OF OPHTHALMIC DEVICES BASED ON PHOTO-INDUCED STEP GROWTH POLYMERIZATION”, 60/869,817 filed Dec. 13, 2006 (entitled “Actinically Curable Silicone Hydrogel Copolymers and Uses thereof”), 60/896,325 filed Mar. 22, 2007 (“Prepolymers with Dangling Polysiloxane-Containing Polymer Chains”), 60/896,326 filed Mar. 22, 2007 (“Silicone-Containing Prepolymers with Dangling Hydrophilic Polymeric Chains”), which are incorporated herein by references in their entireties.

In one aspect, the matrix-forming material comprises a water-soluble crosslinkable poly(vinyl alcohol) prepolymer that is actinically-crosslinkable. In another aspect, the water-soluble crosslinkable poly(vinyl alcohol) prepolymer is a polyhydroxyl compound described in U.S. Pat. Nos. 5,583,163 and 6,303,687 and has a molecular weight of at least about 2,000 and comprises from about 0.5 to about 80%, based on the number of hydroxyl groups in the poly(vinyl alcohol), of units of the formula I-III:

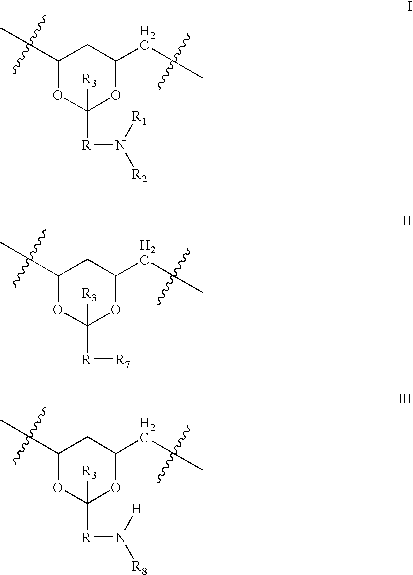

In formula I, II and III, the molecular weight refers to a weight average molecular weight, Mw, determined by gel permeation chromatography.

In formula I, II and III, R₃ can be hydrogen, a C₁-C₆ alkyl group or a cycloalkyl group.

In formula I, II and III, R can be alkylene having up to 8 carbon atoms or up to 12 carbon atoms, and can be linear or branched. Suitable examples include octylene, hexylene, pentylene, butylene, propylene, ethylene, methylene, 2-propylene, 2-butylene and 3-pentylene. Lower alkylene R can be up to 6 or up to 4 carbon atoms. In one aspect, R is methylene or butylene.

In the formula I, R₁ can be hydrogen or lower alkyl having up to seven, in particular up to four, carbon atoms. In the formula I, R₂ can be an olefinically unsaturated, electron-withdrawing, crosslinkable radical having up to 25 carbon atoms. In one aspect, R₂ can be an olefinically unsaturated acyl radical of the formula R₄—CO—, where R₄ is an olefinically unsaturated, crosslinkable radical having 2 to 24, 2 to 8, or 2 to 4 carbon atoms.

The olefinically unsaturated, crosslinkable radical R₄ can be, for example ethenyl, 2-propenyl, 3-propenyl, 2-butenyl, hexenyl, octenyl or dodecenyl. In one aspect, —C(O)R₄ is ethenyl or 2-propenyl so that the —C(O)R₄ is the acyl radical of acrylic acid or methacrylic acid.

In formula II, R₇ can be a primary, secondary or tertiary amino group or a quaternary amino group of the formula N⁺(R′)₃X⁻, where each R′ is, independently, hydrogen or a C₁-C₄ alkyl radical, and X is a counterion such as, for example, HSO₄⁻, F⁻, Cl⁻, Br⁻, I⁻, CH₃ COO⁻, OH⁻, BF⁻, or H₂PO₄⁻. In one aspect, the R₇ is amino, mono- or di(lower alkyl)amino, mono- or diphenylamino, (lower alkyl)phenylamino or tertiary amino incorporated into a heterocyclic ring, for example —NH₂, —NH—CH₃, —N(CH₃)₂, —NH(C₂H₅), —N(C₂H₅)₂, —NH(phenyl), —N(C₂H₅)phenyl or

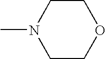

In formula III, R₈ can be a radical of a monobasic, dibasic or tribasic, saturated or unsaturated, aliphatic or aromatic organic acid or sulfonic acid. In one aspect, R₈ is derived from chloroacetic acid, succinic acid, glutaric acid, adipic acid, pimelic acid, maleic acid, fumaric acid, itaconic acid, citraconic acid, acrylic acid, methacrylic acid, phthalic acid, or trimellitic acid.

The term “lower” in connection with radicals and compounds denotes, unless defined otherwise, radicals or compounds having up to 7 carbon atoms. Lower alkyl has, in particular, up to 7 carbon atoms, and includes, for example, methyl, ethyl, propyl, butyl or tert-butyl. Lower alkoxy has, in particular, up to 7 carbon atoms, and includes, for example, methoxy, ethoxy, propoxy, butoxy or tert-butoxy.

In the formula N⁺(R′)₃X⁻, R′ is preferably hydrogen or C₁-C₃ alkyl, and X is halide, acetate or phosphite, for example —N⁺(C₂H₅)₃CH₃COO⁻, —N⁺(C₂H₅)₃Cl⁻, and —N⁺(C₂H₅)₃H₂PO₄

In one aspect, the prepolymer is a water-soluble crosslinkable poly(vinyl alcohol) having a molecular weight of at least about 2,000 and is from about 0.5 to about 80%, from 1 to 50%, from 1 to 25%, or from 2 to 15%, based on the number of hydroxyl groups in the poly(vinyl alcohol), of units of the formula I, wherein R is lower alkylene having up to 6 carbon atoms, R₁ is hydrogen or lower alkyl, R₃ is hydrogen, and R₂ is a radical of formula (IV) or (V).

—CO—NH—(R₅—NH—CO—O)_{q}—R₆—O—CO—R₄ (IV)

—[CO—NH—(R₅—NH—CO—O)_{q}—R₆—O]ₚ—CO—R₄ (V)

in which p and q, independently of one another, are zero or one, and R₅ and R₆, independently of one another, are lower alkylene having 2 to 8 carbon atoms, arylene having 6 to 12 carbon atoms, a saturated bivalent cycloaliphatic group having 6 to 10 carbon atoms, arylenealkylene or alkylenearylene having 7 to 14 carbon atoms or arylenealkylenearylene having 13 to 16 carbon atoms, and in which R₄ is as defined above.

In one aspect, when p is zero, R₄ is C₂-C₈ alkenyl. In another aspect, when p is one and q is zero, R₆ is C₂-C₆ alkylene and R₄ is C₂-C₈ alkenyl. In a further aspect, when both p and q are one, R₅ is C₂-C₆ alkylene, phenylene, unsubstituted or lower alkyl-substituted cyclohexylene or cyclo hexylene-lower alkylene, unsubstituted or lower alkyl-substituted phenylene-lower alkylene, lower alkylene-phenylene, or phenylene-lower alkylene-phenylene, R₆ is C₂-C₆ alkylene, and R₄ is preferably C₂-C₈ alkenyl.

Crosslinkable poly(vinyl alcohol) comprising units of the formula I, I and II, I and III, or I and II and III can be prepared using techniques known in the art. For example, U.S. Pat. Nos. 5,583,163 and 6,303,687 disclose methods for preparing crosslinkable polymers comprising the units of the formula I, I and II, I and III, or I and II and III.

In another aspect, an actinically-crosslinkable prepolymer is a crosslinkable polyurea as described in U.S. Pat. No. 6,479,587 or in U.S. Published Application No. 2005/0113549 (herein incorporated by reference in their entireties). In one aspect, the crosslinkable polyurea prepolymer has the formula (1):

(CP)-(Q)_{q} (1)

wherein q is an integer of >3, Q is an organic radical that comprises at least one crosslinkable group, CP is a multivalent branched copolymer fragment comprising segments A and U and optionally segments B and T.
wherein: A is a bivalent radical of formula (2):

—NR_{A}-A¹-NR_{A}′— (2)

wherein A¹ is the bivalent radical of —(R¹¹O)ₙ—(R¹²O)ₘ—(R¹³O)ₚ—, a linear or branched C₂-C₂₄ aliphatic bivalent radical, a C₅-C₂₄ cycloaliphatic or aliphatic-cycloaliphatic bivalent radical, or a C₆-C₂₄ aromatic or araliphatic bivalent radical, R¹¹, R¹², and R¹³ are, independently, linear or branched C₂-C₄-alkylene or hydroxy-substituted C₂-C₈ alkylene radicals, n, m and p are, independently, a number from 0 to 100, provided that the sum of (n+m+p) is 5 to 1,000, and R_{A} and R_{A}′ are, independently, hydrogen, an unsubstituted C₁-C₆ alkyl, a substituted C₁-C₆ alkyl, or a direct, ring-forming bond;

### T is a bivalent radical of formula (3):

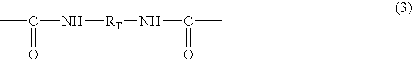

wherein R_{T} is a bivalent aliphatic, cycloaliphatic, aliphatic-cycloaliphatic, aromatic, araliphatic or aliphatic-heterocyclic radical;

### U is a trivalent radical of formula (4):

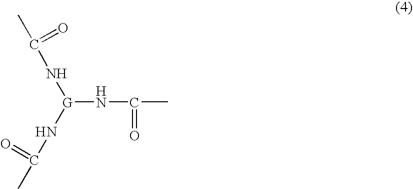

wherein G is a linear or branched C₃-C₂₄ aliphatic trivalent radical, a C₅-C₄₅ cycloaliphatic or aliphatic-cycloaliphatic trivalent radical, or a C₃-C₂₄ aromatic or araliphatic trivalent radical;

### B is a radical of formula (5):

—NR_{B}—B¹—NR_{B}′— (5)

wherein R_{B} and R_{B}′ are, independently, hydrogen, an unsubstituted C₁-C₆ alkyl, a substituted C₁-C₆ alkyl, or a direct, ring-forming bond, B¹ is a bivalent aliphatic, cycloaliphatic, aliphatic-cycloaliphatic, aromatic or araliphatic hydrocarbon radical that is interrupted by at least one amine group —NRₘ—, where Rₘ is hydrogen, a radical Q mentioned above or a radical of formula (6):

Q-CP′— (6)

wherein Q is as defined above, and CP′ is a bivalent copolymer fragment comprising at least two of the above-mentioned segments A, B, T and U; provided that in the copolymer fragments CP and CP′, segment A or B is followed by segment T or U in each case; provided that in the copolymer fragments CP and CP′, segment T or U is followed by segment A or B in each case; provided that the radical Q in formulae (1) and (6) is bonded to segment A or B in each case; and provided that the N atom of —NRₘ— is bonded to segment T or U when Rₘ is a radical of formula (6).

In one aspect, a crosslinkable prepolymer of formula (1) is obtained by introducing ethylenically unsaturated groups into an amine- or isocyanate-capped polyurea, which can be a copolymerization product of a mixture comprising (a) at least one poly(oxyalkylene)diamine, (b) at least one organic poly-amine, (c) optionally at least one diisocyanate, and (d) at least one polyisocyanate. In one aspect, the amine- or isocyanate-capped polyurea is a copolymerization product of a mixture comprising (a) at least one poly(oxyalkylene)diamine, (b) at least one organic di- or poly-amine (preferably triamine), (c) at least one diisocyanate, and (d) at least one polyisocyanate (preferably triisocyanate).

An examples of a poly(oxyalkylene)diamine useful herein includes Jeffamines® having an average molecular weight of, for example, approximately from 200 to 5,000.

The diisocyanate can be a linear or branched C₃-C₂₄ aliphatic diisocyanate, a C₅-C₂₄ cycloaliphatic or aliphatic-cycloaliphatic diisocyanate, or a C₆-C₂₄ aromatic or araliphatic diisocyanate. Examples of diisocyanates useful herein include, but are not limited to, isophorone diisocyanate (IPDI), 4,4′-methylenebis(cyclohexyl isocyanate), toluoylene-2,4-diisocyanate (TDI), 1,6-diisocyanato-2,2,4-trimethyl-n-hexane (TMDI), methylenebis(cyclohexyl-4-isocyanate), methylenebis(phenyl-isocyanate), or hexamethylene-diisocyanate (HMDI).

The organic diamine can be a linear or branched C₂-C₂₄ aliphatic diamine, a C₅-C₂₄ cycloaliphatic or aliphatic-cycloaliphatic diamine, or a C₆-C₂₄ aromatic or araliphatic diamine. In one aspect, the organic diamine is bis(hydroxyethylene)ethylenediamine (BHEEDA).

Examples of polyamines include symmetrical or asymmetrical dialkylenetriamines or trialkylenetetramines. For example, the polyamine can be diethylenetriamine, N-2′-aminoethyl-1,3-propylenediamine, N,N-bis(3-aminopropyl)-amine, N,N-bis(6-aminohexyl)amine, or triethylenetetramine.

The polyisocyanate can be a linear or branched C₃-C₂₄ aliphatic polyisocyanate, a C₅-C₄₅ cycloaliphatic or aliphatic-cycloaliphatic polyisocyanate, or a C₆-C₂₄ aromatic or araliphatic polyisocyanate. In one aspect, the polyisocyanate is a C₆-C₄₅ cycloaliphatic or aliphatic-cycloaliphatic compound containing 3-6 isocyanate groups and at least one heteroatom including oxygen and nitrogen. In another aspect, the polyisocyanate is a compound having a group of formula (7):

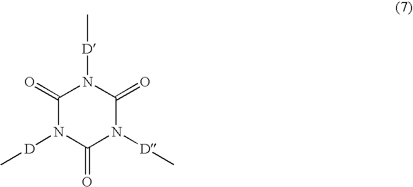

wherein D, D′ and D″ are, independently, a linear or branched divalent C₁-C₁₂ alkyl radical, a divalent C₅-C₁₄ alkylcycloalkyl radical. Examples triisocyanates include, but are not limited to, the isocyanurate trimer of hexamethylene diisocyanate, 2,4,6-toluene triisocyanate, p, p′, p″-triphenylmethane triisocyanate, and the trifunctional trimer (isocyanurate) of isophorone diisocyanate.

In one aspect, the amine- or isocyanate-capped polyurea is an amine-capped polyurea, which may allow the second step reaction to be carried out in an aqueous medium.

When the matrix-forming material comprises a polyurea prepolymer, the prepolymer can be prepared in a manner known to persons skilled in the art using, for example, a two-step process. In the first step, an amine- or isocyanate-capped polyurea is prepared by reacting together a mixture comprising (a) at least one poly(oxyalkylene)diamine, (b) at least one organic di- or poly-amine, (c) at least one diisocyanate, and (d) at least one polyisocyanate. In the second step, a multifunctional compound having at least one ethylenically unsaturated group and a functional group react with the capping amine or isocyanate groups of the amine- or isocyanate-capped polyurea obtained in the first step.

The first step of the reaction can be performed in an aqueous or aqueous-organic medium or organic solvent (e.g, ethyl acetate, THF, isopropanol, or the like). In one aspect, a mixture of water and a readily water-soluble organic solvent, e.g. an alkanol, such as methanol, ethanol or isopropanol, a cyclic ether, such as tetrahydrofuran (THF), or a ketone, such as acetone can be used. In another aspect, the reaction medium is a mixture of water and a readily water-soluble solvent having a boiling point of from 50 to 85° C. or 50 to 70° C. (e.g., such as tetrahydrofuran or acetone).

The reaction temperature in the first reaction step of the process is, for example, from −20 to 85° C., −10 to 50° C., or −5 to 30° C. The reaction times in the first reaction step of the process may vary within wide limits, a time of approximately from 1 to 10 hours, 2 to 8 hours, or 2 to 3 hours having proved practicable.

In one aspect, the prepolymer is soluble in water at a concentration of approximately from 3 to 99% by weight, 3 to 90%, 5 to 60% by weight, or 10 to 60% by weight, in a substantially aqueous solution. In another aspect, the concentration of the prepolymer in solution is from approximately 15 to approximately 50% by weight, approximately 15 to approximately 40% by weight, or from approximately 25% to approximately 40% by weight.

In certain aspects, the prepolymers used herein are purified using techniques known in the art, for example by precipitation with organic solvents, such as acetone, filtration and washing, extraction in a suitable solvent, dialysis or ultrafiltration, ultra-filtration being especially preferred. Thus, the prepolymers can be obtained in extremely pure form, for example in the form of concentrated aqueous solutions that are free, or at least substantially free, from reaction products, such as salts, and from starting materials, such as, for example, non-polymeric constituents.

In one aspect, the purification process for the prepolymers used herein includes ultrafiltration. It is possible for the ultrafiltration to be carried out repeatedly, for example from two to ten times. Alternatively, the ultrafiltration can be carried out continuously until the selected degree of purity is attained. The selected degree of purity can in principle be as high as desired. A suitable measure for the degree of purity is, for example, the concentration of dissolved salts obtained as by-products, which can be determined simply in known manner.

In another aspect, the matrix forming material is a polymerizable composition comprising at least a hydrophilic vinylic monomer including, but not limited to, hydroxyalkyl methacrylate, hydroxyalkyl acrylate, N-vinyl pyrrolidone. The polymerizable composition can further comprise one or more hydrophobic vinylic monomers, crosslinking agent, radical initiators, and other components know to a person skilled in the art. These materials typically require extraction steps.

In another aspect, the polymeric matrix is prepared from silicone-containing prepolymers. Examples of silicone-containing prepolymers are those described in commonly-owned U.S. Pat. Nos. 6,039,913, 7,091,283, 7,268,189 and 7,238,750, and U.S. patent application Ser. No. 09/525,158 filed Mar. 14, 2000 (entitled “Organic Compound”), 11/825,961, 60/869,812 filed Dec. 13, 2006 (entitled “PRODUCTION OF OPHTHALMIC DEVICES BASED ON PHOTO-INDUCED STEP GROWTH POLYMERIZATION”, 60/869,817 filed Dec. 13, 2006 (entitled “Actinically Curable Silicone Hydrogel Copolymers and Uses thereof”), 60/896,325 filed Mar. 22, 2007 (“Prepolymers with Dangling Polysiloxane-Containing Polymer Chains”), 60/896,326 filed Mar. 22, 2007 (“Silicone-Containing Prepolymers with Dangling Hydrophilic Polymeric Chains”).

In another aspect, the matrix forming material is a polymerizable composition comprising at least one silicon-containing vinylic monomer or macromer, or can be any lens formulations for making soft contact lenses. Exemplary lens formulations include without limitation the formulations of lotrafilcon A, lotrafilcon B, confilcon, balafilcon, galyfilcon, senofilcon A, and the like. A lens-forming material can further include other components, such as, a hydrophilic vinylic monomer, crosslinking agent, a hydrophobic vinylic monomer, an initiator (e.g., a photoinitiator or a thermal initiator), a visibility tinting agent, UV-blocking agent, photosensitizers, an antimicrobial agent, and the like. Preferably, a silicone hydrogel lens-forming material used in the present invention comprises a silicone-containing macromer. These materials typically require extraction steps.

Any silicone-containing vinylic monomers can be used in the invention. Examples of silicone-containing vinylic monomers include, without limitation, methacryloxyalkylsiloxanes, 3-methacryloxy propylpentamethyldisiloxane, bis(methacryloxypropyl)tetramethyl-disiloxane, monomethacrylated polydimethylsiloxane, monoacrylated polydimethylsiloxane, mercapto-terminated polydimethylsiloxane, N-[tris(trimethylsiloxy)silylpropyl]acrylamide, N-[tris(trimethylsiloxy)silylpropyl]methacrylamide, and tristrimethylsilyloxysilylpropyl methacrylate (TRIS), N-[tris(trimethylsiloxy)silylpropyl]methacrylamide (“TSMAA”), N-[tris(trimethylsiloxy)silylpropyl]acrylamide (“TSAA”), 2-propenoic acid, 2-methyl-,2-hydroxy-3-[3-[1,3,3,3-tetramethyl-1-[(trimethylsilyl)oxy]disil oxanyl]propoxy]propyl ester (which can also be named (3-methacryloxy-2-hydroxypropyloxy)propylbis(trimethylsiloxy)methylsilane), (3-methacryloxy-2-hydroxypropyloxy)propyltris(trimethylsiloxy)silane, bis-3-methacryloxy-2-hydroxypropyloxypropyl polydimethylsiloxane, 3-methacryloxy-2-(2-hydroxyethoxy)propyloxy)propylbis(trimethylsiloxy)methylsilane, N,N,N′,N′-tetrakis(3-methacryloxy-2-hydroxypropyl)-alpha,omega-bis-3-aminopropyl-polydimethylsiloxane, polysiloxanylalkyl(meth)acrylic monomers, silicone-containing vinyl carbonate or vinyl carbamate monomers (e.g., 1,3-bis[4-vinyloxycarbonyloxy)but-1-yl]tetramethyl-disiloxane; 3-(trimethylsilyl), propyl vinyl carbonate, 3-(vinyloxycarbonylthio)propyl-[tris(trimethylsiloxy)silane], 3-[tris(trimethylsiloxy)silyl]propylvinyl carbamate, 3-[tris(trimethylsiloxy)silyl]propyl allyl carbamate, 3-[tris(trimethylsiloxy)silyl]propyl vinyl carbonate, t-butyldimethylsiloxyethyl vinyl carbonate; trimethylsilylethyl vinyl carbonate, and trimethylsilylmethyl vinyl carbonate). A preferred siloxane-containing monomer is TRIS, which is referred to 3-methacryloxypropyltris(trimethylsiloxy)silane, and represented by CAS No. 17096-07-0. The term “TRIS” also includes dimers of 3-methacryloxypropyltris(trimethylsiloxy)silane. Monomethacrylated or monoacrylated polydimethylsiloxanes of various molecular weight could be used. Dimethacrylated or Diacrylated polydimethylsiloxanes of various molecular weight could also be used. For photo-curable binder polymer, the silicon containing monomers used in the preparation of binder polymer will preferably have good hydrolytic (or nucleophilic) stability.

Any suitable siloxane-containing macromer with ethylenically unsaturated group(s) can be used to produce a silicone hydrogel material. A particularly preferred siloxane-containing macromer is selected from the group consisting of Macromer A, Macromer B, Macromer C, and Macromer D described in U.S. Pat. No. 5,760,100, herein incorporated by reference in its entirety. Macromers could be mono or difunctionalized with acrylate, methacrylate or vinyl groups. Macromers that contain two or more polymerizable groups (vinylic groups) can also serve as cross linkers. Di and triblock macromers consisting of polydimethylsiloxane and polyakyleneoxides could also be of utility. For example one might use methacrylate end capped polyethyleneoxide-block-polydimethylsiloxane-block-polyethyleneoxide to enhance oxygen permeability.

The matrix forming materials used to prepare the polymeric matrix can possess one or more functional groups that are compatible with the bioactive agent. Similarly, the bioactive agent can be modified with one or more functional groups such that when the bioactive agent is incorporated in the polymeric matrix, the bioactive agent does not readily leach from the matrix. In one aspect, the matrix forming material (and the polymeric matrix) comprises at least one ionic group, ionizable group, or a combination thereof. The term “ionic group” is defined herein as any group possessing a charge (positive, negative, or both). The term “ionizable group” is defined as any group that can be converted to an ionic group. For example, an amino group (an ionizable group) can be protonated to produce a positively charged ammonium group (an ionic group).

Examples of anionic, ionic groups include for example C₁-C₆-alkyl substituted with —SO₃H, —OSO₃H, —OPO₃H₂ and —COOH; phenyl substituted with —SO₃H, —COOH, —OH and —CH₂—SO₃H; —COOH; a radical —COOY₄, wherein Y₄ is C₁-C₂₄-alkyl substituted with, for example, —COOH, —SO₃H, —OSO₃H, —OPO₃H₂ or by a radical —NH—C(O)—O-G′ wherein G′ is the radical of an anionic carbohydrate; a radical —CONY₅Y₆ wherein Y₅ is C₁-C₂₄-alkyl substituted with —COOH, —SO₃H, —OSO₃H, or —OPO₃H₂ and Y₆ independently has the meaning of Y₅ or is hydrogen or C₁-C₁₂-alkyl; or —SO₃H; or a salt thereof, for example a sodium, potassium, ammonium or the like salt thereof.

Examples of cationic, ionic groups include for example C₁-C₁₂-alkyl substituted by a radical —NRR′R″⁺An⁻, wherein R, R′ and R′″ are each independently, hydrogen or unsubstituted or hydroxy-substituted C₁-C₆-alkyl or phenyl, and An⁻ is an anion; or a radical —C(O)OY₇, wherein Y₇ is C₁-C₂₄-alkyl substituted by —NRR′R′″⁺An⁻ and is further unsubstituted or substituted for example by hydroxy, wherein R, R′, R′″ and An⁻ are as defined above.

Examples of zwitterionic, ionic groups include a radical —R₁-Zw, wherein R₁ is a direct bond or a functional group, for example a carbonyl, carbonate, amide, ester, dicarboanhydride, dicarboimide, urea or urethane group; and Zw is an aliphatic moiety comprising one anionic and one cationic group each.

In another aspect, the matrix forming materials used to prepare the polymeric matrix can possess one or more hydrophobic groups to increase the hydrophobicity of the polymeric matrix. For example, the matrix forming material can be reacted with a saturated or unsaturated fatty acid prior to polymerization and production of the polymeric matrix. In the alternative, the molecular weight of the matrix forming material can be adjusted in order to increase or decrease the hydrophobicity of the polymeric matrix. In certain instances, when the bioactive agent is a hydrophobic compound, it is desirable to incorporate the bioactive agent in a hydrophobic polymeric matrix to prevent leaching of the agent. The selection of the matrix forming material and bioactive agent with respect to the different types of functional groups that can be used to maximize the incorporation of the bioactive agent into the polymeric matrix will be discussed below.

b. Carrier Agent

In a further aspect, a carrier agent is incorporated in the polymeric matrix. The carrier agent can be covalently attached to the polymer matrix and/or distributed in the polymer matrix to form an interpenetrating polymer network. The carrier agent generally comprises one or more functional groups (e.g., ionic, ionizable, hydrophobic, or any combination thereof). The carrier agent can be used to enhance the incorporation of the bioactive agent into the polymeric matrix. Additionally, the selection of the carrier agent can be used to control the release of the bioactive agent from the polymeric matrix. Not wishing to be bound by theory, it is believed that the carrier agent is weaved throughout the polymeric matrix. This can be accomplished by admixing the carrier agent with the matrix forming material and bioactive agent prior to polymerization. In one aspect, the carrier agent comprises a plurality of ionic or ionizable groups that can impart a charge to a neutral, hydrophobic polymeric matrix. This can be useful when incorporating certain bioactive agent that possess ionic groups. In one aspect, the carrier agents include polycations. In another aspect, the carrier agent comprises a polymer comprising one or more carboxylic acid groups. Specific examples of carrier agents useful herein include, but are not limited to, polyacrylic acid, polymethacrylic acid, polystyrene maleic acid, or a polyethyleneimine.

c. Bioactive Agent

The bioactive agent incorporated in the polymeric matrix is any compound that can prevent a malady in the eye or reduce the symptoms of an eye malady. The bioactive agent can be a drug, an amino acid (e.g., taurine, glycine, etc.), a polypeptide, a protein, a nucleic acid, or any combination thereof. Examples of drugs useful herein include, but are not limited to, rebamipide, ketotifen, olaptidine, cromoglycolate, cyclosporine, nedocromil, levocabastine, lodoxamide, ketotifen, emedastine, naphazoline, ketorolac, or the pharmaceutically acceptable salt or ester thereof. Other examples of bioactive agents include 2-pyrrolidone-5-carboxylic acid (PCA), alpha hydroxyl acids (e.g., glycolic, lactic, malic, tartaric, mandelic and citric acids and salts thereof, etc.), linoleic and gamma linoleic acids, hyaluronan, and vitamins (e.g., B5, A, B6, etc.).

d. Additional Components

In various aspects, additional components can be incorporated into the polymeric matrix. Examples of such components include, but are not limited to, lubricants, ocular salves, thickening agents, or any combination thereof.

Examples of lubricants include without limitation mucin-like materials and hydrophilic polymers. Exemplary mucin-like materials include without limitation polyglycolic acid, polylactides, collagen, hyaluronic acid, and gelatin.

Exemplary hydrophilic polymers include, but are not limited to, polyvinyl alcohols (PVAs), polyamides, polyimides, polylactone, a homopolymer of a vinyl lactam, a copolymer of at least one vinyl lactam in the presence or in the absence of one or more hydrophilic vinylic comonomers, a homopolymer of acrylamide or methacrylamide, a copolymer of acrylamide or methacrylamide with one or more hydrophilic vinylic monomers, and mixtures thereof.

In one aspect, the vinyl lactam referred to above has a structure of formula (VI)

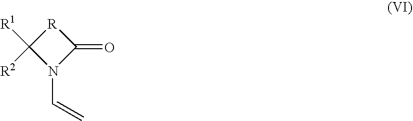

wherein

### R is an alkylene di-radical having from 2 to 8 carbon atoms,

R₁ is hydrogen, alkyl, aryl, aralkyl or alkaryl, preferably hydrogen or lower alkyl having up to 7 and, more preferably, up to 4 carbon atoms, such as, for example, methyl, ethyl or propyl; aryl having up to 10 carbon atoms, and also aralkyl or alkaryl having up to 14 carbon atoms; and

### R₂ is hydrogen or lower alkyl having up to 7 and, more preferably, up to 4 carbon atoms, such as, for example, methyl, ethyl or propyl.

Some N-vinyl lactams corresponding to the above structural formula (V) include N-vinyl-2-pyrrolidone, N-vinyl-2-piperidone, N-vinyl-2-caprolactam, N-vinyl-3-methyl-2-pyrrolidone, N-vinyl-3-methyl-2-piperidone, N-vinyl-3-methyl-2-caprolactam, N-vinyl-4-methyl-2-pyrrolidone, N-vinyl-4-methyl-2-caprolactam, N-vinyl-5-methyl-2-pyrrolidone, N-vinyl-5-methyl-2-piperidone, N-vinyl-5,5-dimethyl-2-pyrrolidone, N-vinyl-3,3,5-trimethyl-2-pyrrolidone, N-vinyl-5-methyl-5-ethyl-2-pyrrolidone, N-vinyl-3,4,5-trimethyl-3-ethyl-2-pyrrolidone, N-vinyl-6-methyl-2-piperidone, N-vinyl-6-ethyl-2-piperidone, N-vinyl-3,5-dimethyl-2-piperidone, N-vinyl-4,4-dimethyl-2-piperidone, N-vinyl-7-methyl-2-caprolactam, N-vinyl-7-ethyl-2-caprolactam, N-vinyl-3,5-dimethyl-2-caprolactam, N-vinyl-4,6-dimethyl-2-caprolactam, and N-vinyl-3,5,7-trimethyl-2-caprolactam.

The number-average molecular weight Mₙ of the hydrophilic polymer is, for example, greater than 10,000, or greater than 20,000, than that of the matrix forming material. For example, when the matrix forming material is a water-soluble prepolymer having an average molecular weight Mₙ of from 12,000 to 25,000, the average molecular weight Mₙ of the hydrophilic polymer is, for example, from 25,000 to 100000, from 30,000 to 75,000, or from 35,000 to 70,000.

Examples of hydrophilic polymers include, but are not limited to, polyvinyl alcohol (PVA), polyethylene oxide (i.e., polyethylene glycol (PEG)), poly-N-vinyl pyrrolidone, poly-N-vinyl-2-piperidone, poly-N-vinyl-2-caprolactam, poly-N-vinyl-3-methyl-2-caprolactam, poly-N-vinyl-3-methyl-2-piperidone, poly-N-vinyl-4-methyl-2-piperidone, poly-N-vinyl-4-methyl-2-caprolactam, poly-N-vinyl-3-ethyl-2-pyrrolidone, and poly-N-vinyl-4,5-dimethyl-2-pyrrolidone, polyvinylimidazole, poly-N-N-dimethylacrylamide, polyacrylic acid, poly 2 ethyl oxazoline, heparin polysaccharides, polysaccharides, a polyoxyethylene derivative, and mixtures thereof.

A suitable polyoxyethylene derivative is, for example, n-alkylphenyl polyoxyethylene ether, n-alkyl polyoxy-ethylene ether (e.g., TRITON®), polyglycol ether surfactant (TERGITOL®), polyoxyethylenesorbitan (e.g., TWEEN®), polyoxyethylated glycol monoether (e.g., BRIJ®, polyoxyethylene 9 lauryl ether, polyoxyethylene 10 ether, polyoxyethylene 10 tridecyl ether), or a block copolymer of ethylene oxide and propylene oxide (e.g. poloxamers or poloxamines).

In one aspect, the polyoxyethylene derivatives are polyethylene-polypropylene block copolymers, in particular poloxamers or poloxamines, which are available, for example, under the tradename PLURONIC®, PLURONIC-R®, TETRONIC®, TETRONIC-R® or PLURADOT®. Poloxamers are triblock copolymers with the structure PEO-PPO-PEO (where “PEO” is poly(ethylene oxide) and “PPO” is poly(propylene oxide). A considerable number of poloxamers is known, differing merely in the molecular weight and in the PEO/PPO ratio; Examples of poloxamers include 101, 105, 108, 122, 123, 124, 181, 182, 183, 184, 185, 188, 212, 215, 217, 231, 234, 235, 237, 238, 282, 284, 288, 331, 333, 334, 335, 338, 401, 402, 403 and 407. The order of polyoxyethylene and polyoxypropylene blocks can be reversed creating block copolymers with the structure PPO-PEO-PPO, which are known as PLURONIC-R® polymers.

Poloxamines are polymers with the structure (PEO-PPO)₂—N—(CH₂)₂—N—(PPO-PEO)₂ that are available with different molecular weights and PEO/PPO ratios. Again, the order of polyoxyethylene and polyoxypropylene blocks can be reversed creating block copolymers with the structure (PPO-PEO)₂—N—(CH₂)₂—N-(PEO-PPO)₂, which are known as TETRONIC-R® polymers.

Polyoxypropylene-polyoxyethylene block copolymers can also be designed with hydrophilic blocks comprising a random mix of ethylene oxide and propylene oxide repeating units. To maintain the hydrophilic character of the block, ethylene oxide will predominate. Similarly, the hydrophobic block can be a mixture of ethylene oxide and propylene oxide repeating units. Such block copolymers are available under the tradename PLURADOT®.

e. Preparation of Ocular Devices

Described herein are methods for preparing ocular devices. The ocular devices are any devices intended to be placed either on the surface of the eye or implanted within the eye using surgical techniques known in the art. For example, the ocular devices can be a contact lens or an intraocular lens. In one aspect, the method comprises the steps of:

a. admixing a matrix-forming material and a bioactive agent;
b. introducing the admixture produced in step (a) into a mold for making the device;
c. polymerizing the matrix-forming material in the mold to form the device, wherein the bioactive agent interacts with the polymeric matrix and is immobilized in the polymeric matrix produced during the polymerization of the matrix-forming material.

The selection of the bioactive agent and the matrix forming material can vary depending upon, among other things, the particular malady to be treated and the desired release pattern of the bioactive agent. For example, if the bioactive agent has one or more anionic ionic/ionizable groups (e.g., COOH groups), the matrix forming material can have one or more cationic ionic/ionizable groups (e.g., NH₂ groups). Here, an electrostatic interaction occurs between the bioactive agent and the polymeric matrix formed after polymerization. For example, vifilcon, which is a prepolymer comprising a copolymer of 2-hydroxyethyl methacrylate and N-vinyl pyrrolidone, contains COOH (anionic) groups. Thus, bioactive agents with ionic groups or ionizable groups (e.g., amino groups that can be converted to positively charged ammonium groups) can be selected to maximize the interaction between the matrix forming material and the bioactive agent. In the alternative, if the matrix forming material does not possess ionic/ionizable groups, a carrier agent possessing a plurality of ionic/ionizable groups can be used to electrostatically interact with the bioactive agent. For example, nelfilcon, which is a prepolymer of polyvinyl alcohol derivatized with N-formyl methyl acrylamide, does not possess ionic or ionizable groups. Thus, a carrier agent such as, for example, polyacrylic acid or polymethacrylic acid can be used to impart charge to the polymeric matrix and enhance the interaction between the polymeric matrix and the bioactive agent.

Another type of interaction to consider when selecting the bioactive agent and matrix forming material is hydrophobic/hydrophobic interactions. If the particular bioactive agent is hydrophobic, at least a portion of the matrix forming material should also be relatively hydrophobic so that the bioactive agent remains in the polymeric matrix and does not leach. One approach to determining the ability of a bioactive agent to release from the polymeric matrix is to look at the partition coefficient of the bioactive agent between the lens polymers and water. Increasing the hydrophobicity of the polymeric matrix or using a more hydrophobic IPN can result in higher drug loading in the lens.

In one aspect, the selection of the bioactive agent and the matrix forming material can be based upon the water-octanol partition coefficient of the bioactive agent between octanol and water. The octanol-water partition coefficient is expressed as logK_{ow}, where K_{ow} is the ratio of bioactive agent in the octanol and water layers. An octanol-water partition coefficient between 0 and −1 indicates that the bioactive agent is comparably soluble in both octanol and water. A partition coefficient in this range is a good indicator that the bioactive agent will be released from the polymer matrix. As the value of the octanol-water partition coefficient decreases (i.e., becomes more negative), the bioactive agent has a greater affinity for water. The pKa of the bioactive agent (i.e., the pH at which 50% of the bioactive agent is ionized) and the pH of the polymeric matrix (i.e., selection of the matrix forming material and functional groups present on the material) are to be considered when producing the ocular device. In certain aspects, the charged groups on the ionized bioactive agent can be paired with charges in the matrix or in a carrier polymer to aid in retention of the bioactive agent.

By varying the hydrophobicity and/or the number of ionic/ionizable groups present on the matrix forming material (and ultimately the polymeric matrix), it is possible to select and incorporate a wide variety of bioactive agents into the polymeric matrix. Moreover, it is possible to tailor the release pattern of the bioactive agent from the ocular device. This is particularly attractive if it is desirable to have sustained release of the bioactive agent over prolonged periods of time.

In another aspect, the bioactive agent can be covalently attached to the matrix forming material prior to polymerization using techniques known in the art. For example, if the matrix forming material is nefilcon, which is a prepolymer of polyvinyl alcohol, the hydroxyl groups can react with a bioactive agent possessing COOH groups to produce the corresponding ester under the appropriate conditions.

Prior to polymerization, the matrix forming material, the bioactive agent, and other optional components (e.g., carrier agents) are intimately mixed using techniques known in the art. The components can be mixed in dry form or in solution. In the case when a solution is used, it is desirable to use water and avoid using organic solvents that may require subsequent purification steps to remove residual solvent. Depending upon the selection of the bioactive agent and the matrix forming material, the pH can be varied to optimize the interaction between the components. During the admixing step, the bioactive agent is thoroughly integrated nor dispersed in the matrix forming material to produce a uniform mixture. This is important, because it ensures that the bioactive agent will be released at consistent concentrations. Thus, the phrase “incorporated within the polymeric matrix” means that the bioactive agent is integrated evenly throughout the entire polymeric matrix and not just localized at particular ocular regions.

After the matrix forming material, bioactive agent, and other optional components have been admixed, the admixture is poured into a mold with a specific shape and size. When the ocular device is a contact lens, the lens can be produced using techniques known in the art. For example, the contact lens can be produced in a conventional “spin-casting mold,” as described for example in U.S. Pat. No. 3,408,429, or by the full cast-molding process in a static form, as described in U.S. Pat. Nos. 4,347,198; 5,508,317; 5,583,463; 5,789,464; and 5,849,810.

Lens molds for making contact lenses are well known in the art. For example, a mold (for full cast molding) generally comprises at least two mold sections (or portions) or mold halves, i.e. first and second mold halves. The first mold half defines a first molding (or optical) surface and the second mold half defines a second molding (or optical) surface. The first and second mold halves are configured to receive each other such that a lens forming cavity is formed between the first molding surface and the second molding surface. The molding surface of a mold half is the cavity-forming surface of the mold and in direct contact with the admixture of matrix forming material and bioactive agent.

Methods of manufacturing mold sections for cast-molding a contact lens are generally well known to those of ordinary skill in the art. The first and second mold halves can be formed through various techniques, such as injection molding or lathing. Examples of suitable processes for forming the mold halves are disclosed in U.S. Pat. Nos. 4,444,711; 4,460,534; 5,843,346; and 5,894,002, which are also incorporated herein by reference.

Virtually all materials known in the art for making molds can be used to make molds for preparing ocular lenses. For example, polymeric materials, such as polyethylene, polypropylene, polystyrene, PMMA, cyclic olefin copolymers (e.g., Topas® COC from Ticona GmbH of Frankfurt, Germany and Summit, New Jersey; Zeonex® and Zeonor® from Zeon Chemicals LP, Louisville, Ky.), or the like can be used. Other materials that allow UV light transmission could be used, such as quartz glass and sapphire.

In one aspect, when the matrix forming material is a fluid prepolymer in the form of a solution, solvent-free liquid, or melt of one or more prepolymers optionally in presence of other components, reusable molds can be used. Examples of reusable molds are those disclosed in U.S. Pat. No. 6,627,124, which is incorporated by reference in their entireties. In this aspect, the fluid prepolymer composition is poured into a mold consisting of two mold halves, the two mold halves not touching each other but having a thin gap of annular design arranged between them. The gap is connected to the mold cavity, so that excess fluid prepolymer composition can flow into the gap. Instead of polypropylene molds that can be used only once, it is possible for reusable quartz, glass, sapphire molds to be used, since, following the production of a lens, these molds can be cleaned rapidly and effectively to remove unreacted materials and other residues, using water or a suitable solvent, and can be dried with air. Reusable molds can also be made of a cyclic olefin copolymer, such as for example, Topas® COC grade 8007-S10 (clear amorphous copolymer of ethylene and norbornene) from Ticona GmbH of Frankfurt, Germany and Summit, New Jersey, Zeonex® and Zeonor® from Zeon Chemicals LP, Louisville, Ky. Because of the reusability of the mold halves, a relatively high outlay can be expended at the time of their production in order to obtain molds of extremely high precision and reproducibility. Since the mold halves do not touch each other in the region of the lens to be produced, i.e. the cavity or actual mold faces, damage as a result of contact is ruled out. This ensures a high service life of the molds, which, in particular, also ensures high reproducibility of the contact lenses to be produced.

Once the admixture is poured into the mold, the matrix forming material is polymerized to produce a polymeric matrix. The techniques for conducting the polymerization step will vary depending upon the selection of the matrix forming material. In one aspect, when the matrix forming material comprises a prepolymer comprising one or more actinically-crosslinkable ethylenically unsaturated groups, the mold containing the admixture can be exposed to a spatial limitation of actinic radiation to polymerize the prepolymer.

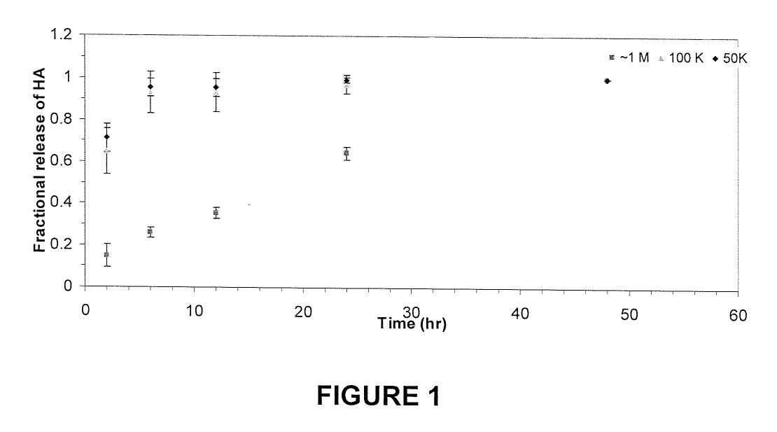

A “spatial limitation of actinic radiation” refers to an act or process in which energy radiation in the form of rays is directed by, for example, a mask or screen or combinations thereof, to impinge, in a spatially restricted manner, onto an area having a well defined peripheral boundary. For example, a spatial limitation of UV radiation can be achieved by using a mask or screen that has a transparent or open region (unmasked region) surrounded by a UV impermeable region (masked region), as schematically illustrated in FIGS. 1-9 of U.S. Pat. No. 6,627,124 (herein incorporated by reference in its entirety). The unmasked region has a well defined peripheral boundary with the unmasked region. The energy used for the crosslinking is radiation energy, especially UV radiation, gamma radiation, electron radiation or thermal radiation, the radiation energy preferably being in the form of a substantially parallel beam in order on the one hand to achieve good restriction and on the other hand efficient use of the energy.

In one aspect, the mold with the admixture is exposed to a parallel beam to achieve good restriction and efficient use of the energy. The time the admixture is exposed to the energy is relatively short, e.g. in less than or equal to 60 minutes, less than or equal to 20 minutes, less than or equal to 10 minutes, less than or equal to 5 minutes, from 1 to 60 seconds, or from 1 to 30 seconds. After polymerization of the matrix forming material, an elaborate matrix is produced where the bioactive agent and other components are meshed in the matrix.

In one aspect, if the ocular device is produced solvent-free from a pre-purified prepolymer, then it is not necessary to perform subsequent purification steps such as extraction. This is because the prepolymer does not contain any undesirable, low molecular weight impurities. One problem associated with extraction is that this process is non-selective in its nature. Anything that is soluble in the employed solvent (e.g., the bioactive agent) and is capable of leaching out the ocular device can be extracted. Additionally, in the extraction process, the device is swollen so that any unbound moieties can be easily removed.

Using the techniques described herein, ocular devices can be produced in a very simple and efficient way compared to prior art techniques. This is based on many factors. First, the starting materials can be acquired or produced inexpensively. Secondly, when the matrix forming materials are prepolymers, the prepolymers are stable so that they can undergo a high degree of purification. Therefore, after polymerization, the ocular device does not require subsequent purification, such as in particular complicated extraction of unpolymerized constituents. Thus, when the ocular device is a contact lens, the ocular device can be directly transformed in the usual way, by hydration, into a ready-to-use contact lens using techniques known in the art. Furthermore, polymerization can be conducted solvent-free or in aqueous solution, so that a subsequent solvent exchange or a hydration step is not necessary. Finally, in the case of photo-polymerization, a short period of time is required, thus the production process can be set up in an extremely economic and efficient way.

The ocular device can be removed from the mold using techniques known in the art. After removal from the mold, the ocular device can be sterilized by autoclaving using techniques known in the art.

When the ocular device is a contact lens, the contact lens can be packaged in packaging solutions known in the art. The packaging solution is ophthalmically compatible, meaning that an ocular device contacted with the solution is generally suitable and safe for direct placement on or in the eye without rinsing. A packaging solution of the invention can be any water-based solution that is used for the storage of ocular devices. Typical solutions include, without limitation, saline solutions, other buffered solutions, and deionized water. In one aspect, the packaging solution is saline solution containing salts including one or more other ingredients including, but not limited to, suitable buffer agents, tonicity agents, water-soluble viscosity builders, surfactants, antibacterial agents, preservatives, and lubricants (e.g., cellulose derivatives, polyvinyl alcohol, polyvinyl pyrrolidone).

The pH of a packaging solution should be maintained within the range of about 6.0 to 8.0, preferably about 6.5 to 7.8. Examples of physiologically compatible buffer systems include, without limitation, acetates, phosphates, borates, citrates, nitrates, sulfates, tartrates, lactates, carbonates, bicarbonates, tris, tris derivatives, and mixtures thereof. The amount of each buffer agent is the amount necessary to be effective in achieving a pH of the composition of from 6.0 to 8.0. The pH can be adjusted accordingly depending upon the bioactive agent incorporated within the polymeric matrix of the ocular device. For example, the pH of the packaging solution can be tailored such that little to no bioactive agent inadvertently leaches from the polymeric matrix.

The aqueous solutions for packaging and storing ocular devices can also be adjusted with tonicity adjusting agents in order to approximate the osmotic pressure of normal lacrimal fluids. The solutions are made substantially isotonic with physiological saline alone or in combination with sterile water and made hypotonic. Correspondingly, excess saline may result in the formation of a hypertonic solution, which will cause stinging and eye irritation. Similar to pH, the saline concentration can be adjusted accordingly depending upon the bioactive agent incorporated within the polymeric matrix of the ocular device. For example, the saline concentration can be adjusted to minimize the leaching of bioactive agent from the polymeric matrix.

Examples of suitable tonicity adjusting agents include, but are not limited to, sodium and potassium chloride, dextrose, glycerin, calcium and magnesium chloride. These agents are typically used individually in amounts ranging from about 0.01 to 2.5% (w/v) and preferably, form about 0.2 to about 1.5% (w/v). In one aspect, the tonicity agent will be employed in an amount to provide a final osmotic value of 200 to 400 mOsm/kg, between about 250 to about 350 mOsm/kg, and between about 280 to about 320 mOsm/kg.

Examples of preservatives useful herein include, but are not limited to, benzalkonium chloride and other quaternary ammonium preservative agents, phenylmercuric salts, sorbic acid, chlorobutanol, disodium edetate, thimerosal, methyl and propyl paraben, benzyl alcohol, and phenyl ethanol.

Surfactants can be virtually any ocularly-acceptable surfactant including non-ionic, anionic, and amphoteric surfactants. Examples of surfactants include without limitation poloxamers (e.g., Pluronic® F108, F88, F68, F68LF, F127, F87, F77, P85, P75, P104, and P84), poloamines (e.g., Tetronic® 707, 1107 and 1307, polyethylene glycol esters of fatty acids (e.g., Tween® 20, Tween® 80), polyoxyethylene or polyoxypropylene ethers of C₁₂-C₁₈ alkanes (e.g., Brij® 35), polyoxyethyene stearate (Myrj® 52), polyoxyethylene propylene glycol stearate (Atlas® G 2612), and amphoteric surfactants under the tradenames Mirataine® and Miranol®.

In one aspect, the packaging solution is an aqueous salt solution having an osmolarity of approximately from 200 to 450 milliosmol per 1000 mL (unit: mOsm/L), approximately from 250 to 350 mOsm/L, and approximately 300 mOsm/L. In other aspects, the packaging solution can be a mixture of water or aqueous salt solution with a physiologically tolerable polar organic solvent, such as, for example, glycerol.

The ocular devices used herein can be stored in any container typically used to store such devices. When the ocular lens is a contact lens, contact lens containers useful herein include are blister packages in various forms.

### II. Methods of Use

The ocular devices described herein can be used to deliver bioactive agents to the eye of a subject. In one aspect, the method comprises contacting the eye of the subject with the ocular devices described herein, wherein one or more tear components releases the bioactive agent from the device. As described above, the ocular devices can be contact lenses that can be applied directly to the surface of the eye. In the alternative, the ocular device can be surgically inserted in the eye. Both of these embodiments fall under the definition of “contacting the eye.”

When the ocular device is contacted with one or more tear components, the bioactive agent is released from the polymeric matrix at a desired rate. The term “tear component” is any biological agent present in the eye or produced by the eye. Tear components are generally any components that would be found in human blood. Examples of tear components include, but are not limited to, lipids, phospholipids, membrane bound proteins, proteins (e.g., albumin, lysozyme, lactoferrin), and salts.

Depending upon the bioactive agent and the matrix forming material used to produce the polymeric matrix, it is possible tailor or design the controlled release of the bioactive agent from the ocular device over extended periods of time. For example, if a drug possessing COOH groups, which is an anionic ionizable group, is incorporated or immobilized in the polymeric matrix, one or more positively-charged proteins present in or produced by the eye (e.g., lysozyme, lactoferrin) can interact with the drug and cause the release of the drug from the polymeric matrix. Here, the positively-charged proteins trigger the release of the drug from the ocular device. Although some release of the bioactive agent from the ocular device is due to passive diffusion (i.e., no external energy required to release the bioactive agent) or eye blink-activated diffusion (i.e., a diffusion process where the eye blinks provide energy to facilitate diffusion of the bioactive agent from the polymer matrix) is possible, it is minimized so that the release of the bioactive agent is caused by one or more tear components interacting with the bioactive agent and/or the polymeric matrix. In the example above, the positively-charged protein released the drug by forming an electrostatic or ionic interaction with the drug. However, other mechanisms are contemplated for releasing the bioactive agent from the polymeric matrix by the tear component including, but not limited to, enzymatic cleavage of a bioactive agent covalently bonded to the polymeric matrix, hydrogen bonding between the bioactive agent and the tear component, and hydrophobic/hydrophobic interactions between the bioactive agent and one or more tear components.

As described above, the release pattern of the bioactive agent can be specifically designed by selecting particular bioactive agents and matrix forming materials used to produce the polymeric matrix. It is also contemplated that the bioactive agent can be modified so that the modified bioactive agent interacts specifically with one or more tear components. For example, if one or more lipids are present in high concentration in the eye, the bioactive agent can be modified with hydrophobic groups to enhance the interaction between the bioactive agent and the lipids, which can ultimately enhance the release of the bioactive agent. The release pattern of the bioactive agent can vary. In one aspect, the release pattern comprises an initial release of bioactive agent (i.e., burst) followed by sustained release of bioactive agent over an extended period of time. The ocular device can release the bioactive agent from 6 hours to 30 days. In another aspect, the ocular device can release the bioactive agent at a controlled rate of 24 hours. Alternatively, the bioactive agent or a portion thereof is not released but remains in the polymeric matrix until it is released by one or more tear components. The interaction between the bioactive agent and polymeric matrix controls the release pattern of the bioactive agent. As described above, factors such as, for example, the pH of the polymeric matrix, the pKₐ of the bioactive agent, and the partitioning of the bioactive agent between hydrophobic and aqueous sections of the polymeric matrix contribute to the controlled release of the bioactive agent.

Additionally, the factors described above can be used to control the amount of bioactive agent that is incorporated in the polymeric matrix and ultimately the ocular device. The amount of bioactive agent that be incorporated into the ocular device and released can vary. Dosing is dependent on severity and responsiveness of the condition to be treated. In the case when the ocular device is a contact device, there is enough bioactive agent present in the device to provide sustained release from several hours up to 30 days, with 24 hours being the preferred. Persons of ordinary skill can easily determine optimum dosages, dosing methodologies and repetition rates.

### EXAMPLES

The following examples are put forth so as to provide those of ordinary skill in the art with a complete disclosure and description of how the compounds, compositions, and methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### I. Cromolyn Sodium

a. Cromolyn Sodium: Drug Loaded Via Absorption into the Dailies Matrix

Cromolyn sodium was strongly absorbed by the Dailies matrix. The amount absorbed from a 4% concentration (equivalent to an ophthalmic solution) soak solution was on the order of 1 mg. Approximately 100 μg was released passively during a short burst period, leaving some 900 μg for release by trigger mechanism. Following passive diffusion, trigger release (using a vortex eye model) resulted in significant release.

b. Cromolyn Sodium: Drug Loaded Directly into the Nelfilcon Macromer

A mixture of Nelfilcon and cromolyn sodium was polymerised to form a membrane, and 1.5 cm diameter discs were cut out and the release profile examined. The release profile of the directly loaded and the absorbed drug described above were compared. Direct loading levels were much lower (approx. 20 μg per lens) than the 1 mg per lens absorbed from a 4% solution. The directly loaded drug had the advantage of achieving virtually zero passive release due to the affinity of the drug to the matrix but again showed very significant triggered release with the in eye model.

II. Ketotifen Fumarate

a. Ketotifen Fumarate: Drug Loaded Via Absorption into the Dailies Matrix

Ketotifen fumarate was used at much lower levels in ophthalmic solution (0.025%) than cromolyn sodium, which was reflected in the uptake experiments. Ketotifen fumarate was absorbed from a 0.025% solution into the lens at a level of 35 μg, with a modest amount released during a short burst period, leaving approximately 30 μg retained in the matrix. This is a very significant payload in relation to daily requirements. Ketotifen fumarate showed enhanced triggered release susceptibility with a vortex eye model relative to passive diffusion. In terms of trigger release, albumin showed little effect but positively charged proteins such as lysozyme showed a significant enhanced effect. The amount of ketotifen fumarate released by triggered release in the vortex eye model from a single lens loaded from a 0.025% solution would be adequate for daily requirements.

b. Ketotifen Fumarate: Drug Loaded Directly into the Nelfilcon Macromer

A mixture of Nelfilcon and ketotifen fumarate was polymerised to form a membrane, and 1.5 cm diameter discs were cut out and the release profile examined. The release profile of the directly loaded and the absorbed drug described above were compared. As with cromolyn sodium, the matrix distribution of the drug loaded directly into the polymer matrix produced differences in release behaviour compared to the absorbed drug. In summary, passive diffusion comes rapidly to equilibrium (within a three hour period) leaving matrix-bound drug, but subsequent trigger release (using a vortex eye model) provided very effective further release, which was enhanced by positively charged tear protein such as lysozyme.

### III. ASM981

a. Direct Loading of ASM981 into the Nelfilcon Macromer

The addition of Pimecrolimus (SDZ ASM981), which is synthesized by Novartis Pharma, in solution form to Nelfilcon macromer increases the liquid content of the macromer. Simple addition of the ASM981 consequently diluted the macromer and photopolymerisation produced wet structurally incoherent product. A membrane composed of 1% ASM981 was prepared by adding 1 g of the ASM981 solution to 5 g of nelfilcon macromer, vortexing for approximately 5 minutes, and removing the lid of the vial to remove excess water. The mass of the ASM981-loaded macromer was allowed to return to its original 5 g. This was conveniently achieved by leaving the mixture overnight on a flatbed shaker under a nitrogen blanket. The mixture was then placed in a membrane mould and polymerised under a static UV lamp. The mixture was successfully polymerised to form a coherent membrane, and the resultant membrane was opaque in appearance. Aqueous passive and agitated release has been examined but, and no release was observed.

### IV. Hyaluronan

a. Direct Loading of Hyaluronan into the Nelfilcon Macromer

Using the techniques above, a mixture of Nelfilcon and varying amounts of hyaluronan was polymerised to form a membrane. The amount of hyaluronan loaded into the Nelfilcon macromer was 2, 6.5, and 40 mg hyaluronan/g nelfilcon (30% by weight aqueous solution). The hyaluronan used was approximately 50 kDa, 100 kDa, and 1 million Da.

b. Characterization of the Hyaluronan Membrane

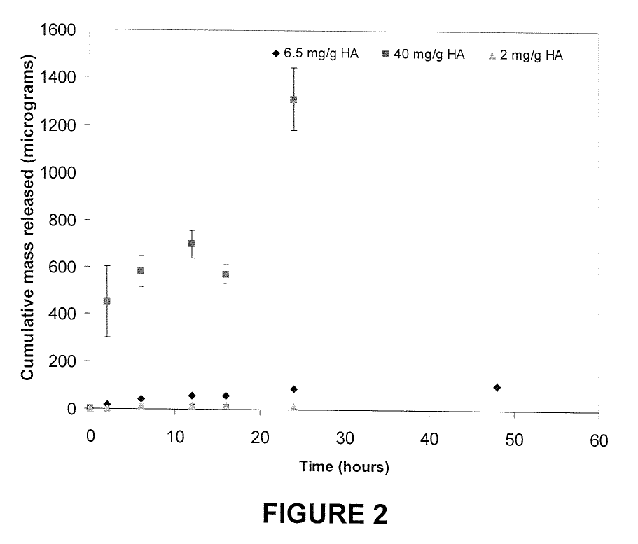

The release of hyaluronan from the membrane was investigated by varying the amount and length of the hyaluronan incorporated into the matrix. Release studies were performed by placing each lens in a solution of 5 mL of artificial lacrimal solution at 35° C. FIG. 1 shows the release pattern of hyaluronan (loading of 6.5 mg HA/g nelfilcon) at various molecular weights. FIG. 1 reveals that the high molecular weight hyaluronan (˜1 M Da) has a relatively constant release rate from 2 to 48 hours. FIG. 2 shows that by increasing the amount of hyaluronan significantly affects the release of the hyaluronan from the matrix.

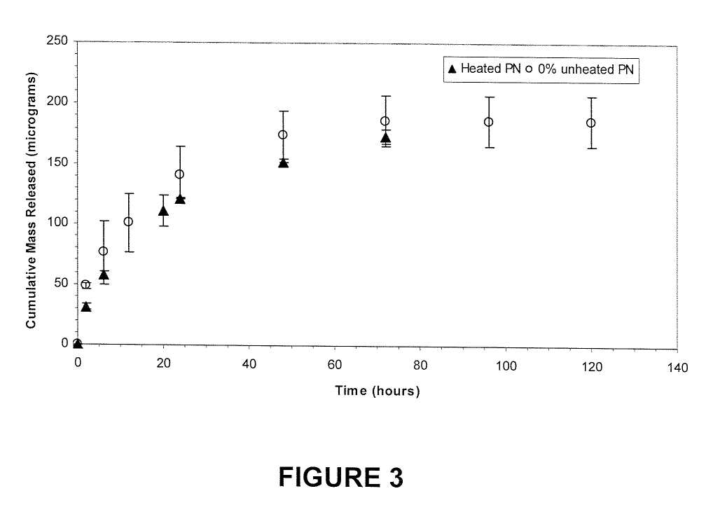

Heat stability studies were also performed on the membranes. A lens prepared from 6.5 mg/mL loading of 1 M Da hyaluronan was placed in a tube of 6.5 mg/mL solution of hyaluronan at a pH of 11. The tube was sealed with a total volume of 0.8 mL, and the solution was heated at 120° C. for 40 minutes. FIG. 3 shows the amount of hyaluronan released over time. FIG. 3 shows that the matrix can protect the hyaluronan from degradation since the release curve is similar to that of the release of hyaluronan from the matrix that is not heated.

### V. Vortex Eye Model

The vortex model is the in vitro in-eye release model described in commonly owned copending US Patent Application Publication No. 2006/0251696 A1 (herein incorporated by reference in its entirety). The experiment is carried out as follows. A contact lens is first blotted dry and immediately is carefully placed into 100 microliter of an extraction medium in an tube (e.g., a centrifuge tube, a scintillation vial, or preferably an Eppendorf microtube) and the microtube is agitated for fifteen seconds using, e.g., a Vibrex vortex mixer. At the end of one hour period, the tube is again agitated using, e.g., a Vibrex vortex mixer, for a further fifteen seconds. The extraction medium is removed from the Eppendorf microtube and 100 microliter of a fresh extraction medium is added. Extraction samples are stored at 25° C. between agitation procedures. The concentration of a guest material extracted out of a lens can be determined according to any methods known to a person skilled in the art.

### VI. Triggered Release by Lysozyme

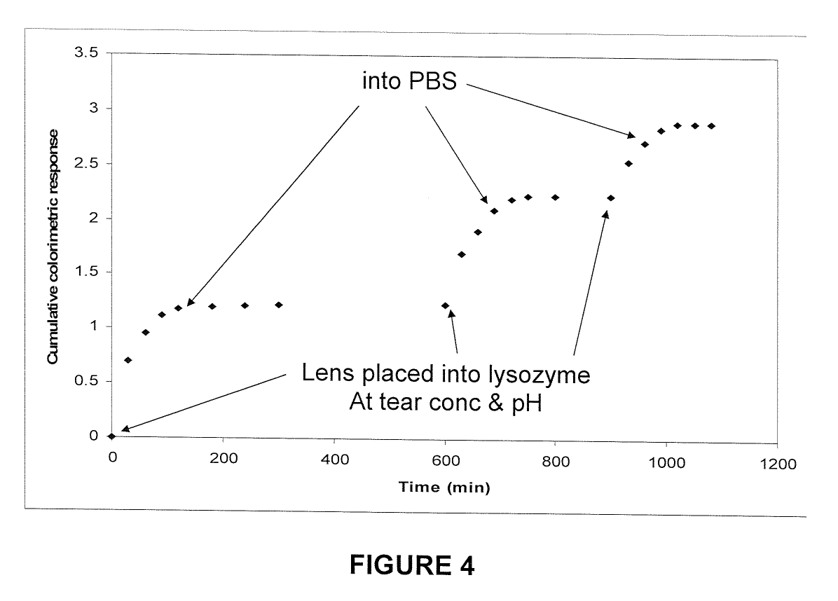

FIG. 4 shows the release pattern of Rose Bengal from Nelfilcon lenses placed in saline solutions (PBS) and lysozyme. Referring to FIG. 4, when the lens is initially placed in a solution of lysozyme (minute zero), the Rose Bengal is released steadily. When the lens is placed in a PBS solution with no lysozyme (approximately minute 150), the little to no Rose Bengal was released. Similar release patterns were observed when the lenses were stored in PBS for eight weeks. In summary, the Nelfilcon lens loaded with Rose Bengal is stable in saline solutions for extended periods of time yet the lens releases the Rose Bengal upon insertion into a solution of lysozyme, which is a tear component.

Throughout this application, various publications are referenced. The disclosures of these publications in their entireties are hereby incorporated by reference into this application in order to more fully describe the compounds, compositions and methods described herein.

Various modifications and variations can be made to the compounds, compositions and methods described herein. Other aspects of the compounds, compositions and methods described herein will be apparent from consideration of the specification and practice of the compounds, compositions and methods disclosed herein. It is intended that the specification and examples be considered as exemplary.

What is claimed:

1. An ocular device comprising a polymeric matrix and a bioactive agent incorporated within the polymeric matrix of the bioactive agent, wherein the ocular device is capable of being induced by one or more tear components to release the bioactive agent from the polymeric matrix when in contact with tears in an eye.

2. The device of claim 1, wherein the bioactive agent is immobilized within the polymeric matrix by an electrostatic interaction, a hydrophobic/hydrophobic interaction, covalently attached to the polymeric matrix, or any combination thereof.

3. The device of claim 1, wherein the polymer matrix is produced by the polymerization of a composition comprising a prepolymer.

4. The device of claim 3, wherein the prepoloymer is water-soluble.

5. The device of claim 3, wherein the prepolymer comprises a water-soluble crosslinkable polyvinyl alcohol prepolymer; a water-soluble vinyl group-terminated polyurethane; a derivative of a polyvinyl alcohol, polyethyleneimine or polyvinyl amine; a water-soluble crosslinkable polyurea prepolymer; a crosslinkable polyacrylamide; a crosslinkable statistical copolymer of vinyl lactam, methyl methacrylate and a comonomer; a crosslinkable copolymer of vinyl lactam, vinyl acetate and vinyl alcohol; a polyether-polyester copolymer with crosslinkable side chains; a branched polyalkylene glycol-urethane prepolymer; a polyalkylene glycol-tetra(meth)acrylate prepolymer; a crosslinkable polyallyl amine gluconolactone prepolymer, or any mixture thereof.

6. The device of claim 3, wherein prepolymer comprises a silicone-containing prepolymer.

7. The device of claim 3, wherein the prepolymer comprises an acrylated polyvinyl alcohol.

8. The device of claim 3, wherein the prepolymer comprises polyvinyl alcohol derivatized with N-formyl methyl acrylamide.

9. The device of claim 1, wherein the bioactive agent and the polymeric matrix comprises at least one ionic group, ionizable group, or a combination thereof.

10. The device of claim 1, wherein the bioactive agent comprises a drug, an amino acid, a polypeptide, a protein, a nucleic acid, or any combination thereof.

11. The device of claim 1, wherein the bioactive agent comprises a drug, wherein the drug comprises rebamipide, olaptidine, cromoglycolate, cromolyn sodium, cyclosporine, nedocromil, levocabastine, lodoxamide, ketotifen, pimecrolimus, hyaluronan, or the pharmaceutically acceptable salt or ester thereof.

12. The device of claim 1, wherein the device further comprises a carrier agent incorporated in the polymeric matrix, wherein the carrier agent comprises at least one ionic group, ionizable group, or a combination thereof.

13. The device of claim 11, wherein the carrier agent comprises a polymer comprising one or more carboxylic acid groups.

14. The device of claim 11, wherein the carrier agent comprises polyacrylic acid, polymethacrylic acid, or a polyethyleneimine.

15. The device of claim 1, wherein the ocular device is characterized by having capability of being stored in a packaging solution for an extended period of time without leaching to a significant extent.

16. The device of claim 1, wherein the bioactive agent is released from the polymeric matrix from 6 hours to 30 days.

17. The device of claim 1, wherein the device comprises a contact lens or an intraocular lens.

18. A process for making an ocular device comprising the steps of:
a. admixing a matrix-forming material and a bioactive agent;
b. introducing the admixture produced in step (a) into a mold for making the device;
c. polymerizing the matrix-forming material in the mold to form the device, wherein the bioactive agent interacts with the polymeric matrix and is immobilized in the polymeric matrix produced during the polymerization of the matrix-forming material.

19. The process of claim 18, wherein the matrix forming material comprises a prepolymer.

20. The process of claim 18, wherein the device produced by the process is not subjected to an extraction process.

21. A device made by the process of claim 18.

22. A method for delivering a bioactive agent to a subject, comprising contacting the eye of the subject with the device of claim 1, wherein one or more tear components releases the bioactive agent from the device.
